(19) United States
(12) Patent Application Publication
(10) Pub. No.: US 2008/0081070 A1
(43) Pub. Date: Apr. 3, 2008

(54) PHARMACEUTICAL FORMULATION WITH ENHANCED SOLUBILITY FOR THE DELIVERY OF CORTICOSTEROIDS

(75) Inventors: Glynn Wilson, Duluth, GA (US); Gerhard Renner, Darmstadt (DE); Hema Ravishankar, Mumbai (IN); Preeti Patil, Mumbai (IN)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Auriga Laboratories, Inc.

(21) Appl. No.: 11/680,276

(22) Filed: Feb. 28, 2007

Related U.S. Application Data
(60) Provisional application No. 60/825,855, filed on Sep. 15, 2006.

(51) Int. Cl. A61K 9/32; A61K 31/56; A61P 1/00

(52) U.S. Cl. 424/482; 514/169

(57) ABSTRACT

Formulations have been developed to improve the solubility of corticosteroids such as fluticasone proprionate in a composition designed to achieve localized release of the drug in the small intestine and/or colon. In one embodiment, solid dispersions of fluticasone are prepared wherein the drug is blended with or coated onto a highly water soluble substrate such as nonpareil (sugar beads) then coated with a layer of polymer soluble in small intestinal fluid, then coated with an enteric coating. The inner polymer layer controls release of the drug, and the enteric coating, a pH sensitive polymer that is broken down in the ileum and colon, controls localized release of drug at various sites within the gastrointestinal tract. The multilayer pharmaceutical composition can be in the form of pellets, tablets compressed from pellets or pellets packed into capsules. The release profile of the drug can be manipulated by (1) altering size or shape (i.e., surface area) and solubility of the inert substrate; (2) the ratio of drug to polymer, the polymer composition and solubility, the porosity of the polymer; (3) the drug form (i.e., free base or salt, or which salt); and the thickness and/or surface area of the drug/polymer and/or enteric coating. In a preferred embodiment, the composition is administered orally. This may also be packaged to provide for an escalating or tapering dosage.

### CROSS-REFERENCE. TO RELATED APPLICATIONS

The application claims priority to U.S. Ser. No. 60/825,855, filed in the United States Patent and Trademark Office on Sep. 15, 2006.

### FIELD OF THE INVENTION

The present Invention relates to pharmaceutical formulations of corticosteroids such as fluticasone, especially compositions designed to enhance the aqueous solubility of fluticasone propionate in the gastrointestinal tract.

### BACKGROUND OF THE INVENTION

Corticosteroids (GC's) are among the most widely used class of drugs for the treatment of atopic (allergic) diseases. Systemic GC's are most often used as rescue therapy for acute asthma exacerbations and can be given orally, intravenously, or intramuscularly. In addition, topical GC's are widely used in the treatment of asthma, allergic rhinitis, and atopic dermatitis. Orally inhaled GC's are considered first-line controller agents for patients with persistent asthma. One of the most widely used GC's is fluticasone propionate. Fluticasone propionate is used to treat respiratory related illnesses such as asthma, emphysema, respiratory distress syndrome, chronic obstructive pulmonary disease (COPD), chronic bronchitis, cystic fibrosis, acquired immune deficiency syndrome, including AIDS related pneumonia, seasonal or perennial rhinitis, seasonal or perennial allergic and nonallergic (vasomotor) rhinitis, and skin conditions treatable with topical corticosteroids. Like other topical GC's, fluticasone propionate has anti-inflammatory, antipruritic, and vasoconstrictive properties.

Fluticasone propionate is marketed in several different commercial forms, in particular, nasal and pulmonary formulations, topical ointments and other topical formulations. ADVAIR DISKUS® (GlaxoSmithKline, Research Triangle Park, N.C.) is an inhalation powder containing a combination of microfine fluticasone, propionate and salmeterol xinofoate, which is a highly selective beta₂-adrenergic bronchodilator. The dosage form is marketed in three doses of fluticasone propionate: 100 microgram, 250 microgram, and 500 micrograms, FLOVENT DISKUS® (GlaxoSmithKline, Research Triangle Park, N.C.) is an oral inhalation powder of micro-fine fluticasone propionate (50 microgram, 100 microgram, and 250 microgram) in lactose. FLOVENT DISKUS® is indicated for the maintenance treatment of asthma, as prophylactic therapy, and for patients requiring oral corticosteroid therapy for asthma. FLOVENT ROTADISK® (GlaxoSmithKline, Research Triangle Park, N.C.) is an oral inhalation powder of microfine fluticasone propionate (50 microgram, 100 microgram, and 250 microgram) blended with lactose. ROTADISK® is indicated for the maintenance treatment of asthma as prophylactic therapy, and for patients requiring oral corticosteroid therapy for asthma. FLOVENT® (GlaxoSmithKline) is an oral inhalation aerosol of a microcrystalline suspension of fluticasone propionate (44 microgram, 110 microgram, or 220 microgram) in a mixture of two chlorofluorocarbon propellants (trichlorofluoromethane and dichlorodifluoromethane) with lecithin. Each actuation of the inhaler deliver 50, 125, or 250 mcg of fluticasone propionate from the valve, and 44, 110, or 220 mcg, respectively, of fluticasone propionate from the actuator. FLOVENT® is indicated for the maintenance treatment of asthma as prophylactic therapy.

FLONASE® (GlaxoSmithKline) is a nasal spray of an aqueous suspension of microfine fluticasone propionate (50 mcg) administered by means of a metering, atomizing spray pump. FLONASE® nasal spray is indicated for the management of the nasal symptoms of seasonal and perennial allergic and nonallergic rhinitis.

CULTIVATE® (GlaxoSmithKline) is a topical dermatological fluticasone propionate cream or ointment (0.05% and 0.005% concentration). The cream and ointment are a medium potency corticosteroid indicated for the relief of the inflammatory and pruritic manifestations of corticosteroid-responsive dermatoses.

Adverse reactions from the current marketed forms of fluticasone propionate include lymphatic signs and symptoms; cardiovascular palpitations; hypersensitivity reactions, including agioedema, skin rash, edema of the face and tongue; pruritus; urticaria; bronchospasm; wheezing; dyspnea; anaphylaxis/anaphylactoid reactions; otitis media; tonsillitis; rhinorrhea/postnasal drip/nasal discharge; earache; cough; laryngitis; hoarseness/dysphonia; epistaxis; tonsillitis;,nasal signs and symptoms; unspecified oropharyngeal plaques; ear, nose, and throat polyps; sneezing; pain in nasal sinuses; rhinitis; throat constriction; allergic ear, nose, and throat disorders; alteration or loss of sense of taste and/or smell; nasal septal perforation; blood in nasal mucosa; nasal ulcer; voice changes; fluid disturbances; weight gain; goiter; disorders of uric acid metabolism; appetite disturbances; irritation of the eyes; blurred vision; glaucoma; increased intraocular pressure and cataracts; keratitis and conjunctivitis; blepharoconjunctivitis; nausea and vomiting; abdominal pain; viral gastroenteritis; gastroenteritis/colitis; gastrointestinal infections; abdominal discomfort; diarrhea; constipation; appendicitis; dyspepsia and stomach disorder; abnormal liver function; injury; fever; tooth decay; dental problems; mouth irritation; mouth and tongue disorders; cholecystitis; lower respiratory infections; pneumonia; arthralgia and articular rheumatism; muscle cramps and spasms; fractures; wounds and lacerations; contusions and hematomas; burns; musculoskeletal inflammation; bone and cartilage disorders; pain in joint; sprain/strain; disorder/symptoms of neck; muscular soreness/pain; aches and pains; pain in limb; dizziness/giddiness; tremors; hypnagogic effects; compressed nerve syndromes; sleep disorders; paralysis of cranial nerves; migraine; nervousness; bronchitis; chest congestion and/or symptoms; malaise and fatigue; pain; edema and swelling; bacterial infections; fungal infections; mobility disorders; cysts, lumps, and masses; mood disorders; acute nasopharyngitis; dyspnea; irritation due to inhalant; urticaria; rash/skin eruption; disorders of sweat and sebum; sweating; photodermatitis; dermatitis and dermatosis; viral skin infections; eczema; fungal skin infections; pruritus; acne and folliculitis; burning; hypertrichosis; increased erythema; hives; folliculitis; hypopigmentation; perioral dermatitis; skin atrophy; striae; miliaria; pustular psoriasis; urinary infections; bacterial reproductive infections; dysmenorrhea; candidiasis of vagina; pelvic inflammatory disease; vaginitis/vulvovaginitis; and irregular menstrual cycle.

These adverse reactions, typical of corticosteroids as a class, can ultimately limit the use of fluticasone. Therefore, there is a need to develop locally acting formulations of fluticasone with reduced systemic side effects.

Fluticasone propionate is also a promising therapeutic agent for the treatment of inflammatory bowel diseases such as ulcerative colitis and Crohn's disease. The affinity of fluticasone propionate for the human glucocorticoid receptor is 18 times greater than dexamethasone, and almost twice that of becloinethasone-17-monopropionate (the active metabolite of budesonide), the two most widely used corticosteroids for the treatment of inflammatory bowel disease. Studies using oral dosing of radiolabeled fluticasone propionate in humans have demonstrated that fluticasone propionate is highly extracted from plasma and that absorption is low. Oral bioavailability is negligible and the majority (less than 1%) of circulating radioactivity is due to an inactive metabolite. The total blood clearance of fluticasone proprionate is high (average, 1,093 mL/min) and predominantly through the liver. The only circulating metabolite identified in man (17-β-carboxylic acid) has negligible pharmacologic activity in animal studies and is formed through the cytochrome P450 3A4 pathway.

Corticosteroids (e.g., prednisone, prednisolone, hydrocortisone, etc.) have been used for many years to treat patients with moderate to severe Crohn's disease and ulcerative colitis and to treat patients who fail to respond to 5-aminosalicylic acid. Short courses of high-dose systemically administered steroids can often bring a patient into remission; remission is ideally maintained with less toxic drugs. However some patients have a flare up of their disease whenever oral steroids are tapered. Thus, a corticosteroid preparation that is active locally, but not systemically absorbed, would be of great benefit. This is because long-term systemic corticosteroids are associated with serious toxicity. In a small number of patients, even acute therapy with prednisone or prednisolone may cause severe'side effects, particularly neuropsychiatric effects. Currently in the United States, budesonide (marketed under the tradename Entocort® EC by Prometheus Therapeutics and Diagnostics, San Diego, Calif.) is approved for the treatment of “mild to moderately active” Crohn's disease of the ileum and/or ascending colon as well as for the maintenance of remission for up to three months in the same population. Clinical studies show that budesonide has similar efficacy to prednisone, but results in fewer clinical features of hypercortisolism and a greater likelihood of maintaining a normal plasma cortisol level, indicating non-suppression of normal adrenal glucocorticoid activity. Furthermore, in a two-year study, bone mineral density decreased less with budesonide than with prednisone treatment. Budesonide is to some extent absorbed systemically and is in fact associated with some corticosteroid side-effects, albeit less than prednisone.

U.S. Pat. No. 4,985,418 to Richards describes the use of fluticasone propionate in the treatment of bowel diseases when administered by oral or rectal route. In a separate pilot study (de Kaski et al, Gut, 32, 657-661 (1991)) for the treatment of mild and moderately active Crohn's disease, twelve patients received oral fluticasone propionate for three weeks. There was a significant fall in Crohn's disease activity index values over the three week treatment period (193 (84) v 122 (51), p less than 0.01). Leucocyte scan images were improved (seven patients) or unchanged (five patients). There was a significant fall in excretion of injected radioactivity calculated from whole body data (28 (21%) v 1.4 (0.7%), p less than 0.05). There were no changes in plasma cortisol values.

The potency of fluticasone, combined with its negligible oral bioavailability, rapid clearance, and no active metabolites makes it an excellent candidate for the localized treatment of Crohn's disease in the gastrointestinal tract. These properties restrict, the pharmacologic effects of fluticasone to local disease sites within the gastrointestinal tract with the avoidance of systemic side effects normally associated with glucocorticoid steroid therapy. For the treatment of ulcerative colitis and Crohn's disease, the avoidance of systemic effects may enable the drug to be used for maintenance therapy once remission has been achieved.

A limitation on the use of fluticasone proprionate for the treatment of inflammatory bowel disease is poor water solubility (0.14 μg/ml in water) resulting in long dissolution times in gastrointestinal fluids. Improvement in the aqueous solubility of fluticasone propionate solubility would result in increased bioavailability of the drug at sites of inflammation in the small intestine or colon.

A number of efforts have been made to create orally deliverable corticosteroids. WO 97/00512 and U.S. Pat. No. 5,849,327 to Berliner et al. describe pharmaceutical forms for release of active ingredients such as, for example, budesonide in the lower gastrointestinal tract. The pharmaceutical form contains the active ingredients bound in cross linked polysaccharide particles which are microbially degradable and coated with EUDRAGIT® S 100 (copolymer of methylmethacrylate and methacrylic acid). The particles are packed into capsules which may, for example, in turn be coated with EUDRAGIT® S 100. WO 01/68058 to Röhm GmbH & Co. KG describes the use of a multilayer pharmaceutical form which is contains a core with an active pharmaceutical ingredient which may be Budesonide, b) an inner coating of a copolymer or a mixture of copolymers which are composed of 85 to 98% by weight free radical polymerized C1 to C4-alkyl esters of acrylic or methacrylic acid and 15 to 2% by weight methacrylate monomers with a quaternary ammonium group in the alkyl radical, and c) an outer coating of a copolymer which is composed of 75 to 95% by weight free-radical polymerized C1 to C4-alkyl esters of acrylic or methacrylic acid and 5 to 25% by weight methacrylate monomers with a anionic group in the alkyl radical. In a USP release test, the formulation releases for two hours at pH 1.2 and subsequent rebuffering to pH 7.0 releases the contained active ingredient to the extent of less than 5% in the period up to 2.0 hours after the start of the test and to the extent of 30 to 80% after eight hours from the start of the test. The outer coating may be of the EUDRAGIT® FS type.

United States patent Application Publication No. US 2005/0089571 by Beckert et al. describes a pharmaceutical formulation containing a) an inner layer which can optionally be applied to a core, with the active substance budesonide, bound with a polymer or a copolymer with an acid group, b) a middle layer with a polymer covering agent which is soluble in intestinal juice or retardant; and c) an outer layer which is resistant to stomach juice. Typically the binder comprises a (meth)acryate copolymer which comprises 40 to 95% by weight free-radical polymerised units of C1 to C4-alkyl esters of acrylic or methacrylic acid and 5 to 60% by weight (meth)acrylate monomers with an anionic group in alkyl radical is employed.

WO 95/08323 by Dr. Falk Pharma GmbH describes budesonide pellets with a controlled release profile and a process of producing them. To improve the solubility of budesonide, the active ingredient was applied to the pellet cores in an alcohol:water mixture of 0:100 to 20:80 and at least two parts of a water soluble excipient, eg. α-lactose monohydrate, sucrose or monosodium citrate, were added to one part of the mixture, then coated with a two-layer coating of, for example, EUDRAGIT® L, S, RS, and/or RL inside and EUDRAGIT® RS/RL outside. Rudolph et al (Proc. of 4th world meeting ADRITELF/APV/APGI, Florence, 8/11 Apr. 2002), describe improving the dissolution rate of budesonide using a fluid bed coating method using EUDRAGIT® L 30D 55 as ionic polymeric carrier and Kollidon® VA 64 as non-ionic polymeric carrier. US Patent Application Publication No. 2004/0208833 to Hovery et al. describes the preparation of a nanoparticulate suspension of fluticasone proprionate as an approach to improve dissolution. Magee et al (Drug Dev. Ind. Pharm. 2003, 4, 441-450 describe an approach using bile salt/lecithin mixed micelles to improve the solubility of fluticasone, however, this method requires large concentrations of bile salts and lecithin that are not compatible with the multilayer pharmaceutical compositions described herein.

There is still a significant need for pharmaceutical compositions of corticosteroids such as fluticasone proprionate with increased solubility that can achieve high local concentrations of the drug at sites of inflammation in the gastrointestinal tract to improve clinical efficacy while avoiding systemic side effects typical of glucocorticoids that have higher oral bioavailability.

Therefore it is an object of the invention to provide pharmaceutical compositions of corticosteroids such as fluticasone proprionate with high solubility in aqueous solutions that can achieve high local concentrations of the drug at sites of inflammation in the gastrointestinal tract to improve clinical efficacy while avoiding systemic side effects typical of glucocorticoids that have higher oral bioavailability, and methods of making and using thereof.

### SUMMARY OF THE INVENTION

Formulations have been developed to improve the solubility of corticosteroids such as fluticasone proprionate in a composition designed to achieve localized release of the drug in the small intestine and colon. In one embodiment, solid dispersions of fluticasone are prepared wherein the drug is blended with or coated onto a highly water soluble substrate such as nonpareil (sugar beads) which is coated with a layer of polymer soluble in small intestinal fluid, then is coated with an enteric coating. Suitable polymeric carriers include, but are not limited to, Eudragit® L 100-55; Eudragit® L30-D55; and Kollidon® VA 64. The ratio of drug to polymer can be varied to achieve the desired solubility of the drug. The inner polymer layer controls release of the drug, and the enteric coating, a pH sensitive polymer that is broken down in the ileum and colon, controls localized release of drug at various sites within the gastrointestinal tract. The multilayer pharmaceutical composition can be in the form of pellets, tablets compressed from pellets or pellets packed into capsules. The compositions can be formulated to precisely control the release of fluticasone proprionate at different sites within the gastrointestinal tract. This can be achieved by using combinations of drug containing pellets differing in the properties of the polymer coatings with respect to drug release profiles.

The release profile of the drug can be manipulated by (1) altering the size or shape (i.e., surface area) and solubility of the inert substrate; (2) altering the ratio of drug to polymer, the polymer composition and the solubility and/or the porosity of the polymer; (3) altering the drug form (i.e., free base or salt, or which salt); and altering the thickness and/or surface area of the drug/polymer and/or enteric coating.

In a preferred embodiment, the composition is administered orally. The formulation may also be packaged to provide for an escalating or tapering dosage.

### DETAILED DESCRIPTION OF THE INVENTION

### I. Definitions

As used herein “fluticasone” encompasses fluticasone and pharmaceutically acceptable salts thereof; pharmaceutically acceptable, pharmacologically active derivatives of fluticasone and their pharmaceutically acceptable salts; and active metabolites of fluticasone and their pharmaceutically acceptable salts, unless otherwise noted. It is understood that in some cases dosages of derivatives, and metabolites may need to be adjusted.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric acids; and the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic acids.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

A “disorder” includes any condition, illness, disease, or infection.

“Effective amount” or “therapeutically effective amount” means the amount needed, for the desired therapeutic effect and includes any additional amount or overage of active ingredient deemed necessary in the formulation to provide the desired amount upon administration.

The phrase “alleviating a gastrointestinal disorder” means reducing or eliminating one or more symptoms suffered by the patient due to one or more conditions, illnesses, infections, or disease states involving the gastrointestinal tract, including, but not limited to the stomach and/or bowel. Exemplary gastrointestinal disorders include, but are not limited to, ulcer, bowel spasms, abdominal pain, bloating, cramps, inflammation of the stomach and/or intestines, irritable bowel syndrome, Crohn's diseases and inflammatory bowel disease.

“Immediate Release” or “IR” means the therapeutic pharmaceutical composition is provided in a formulation allowing the active agent to begin acting in a therapeutic manner substantially as soon as the agent becomes available in the body and/or bloodstream of the patient.

A “delayed release dosage form” is one that releases a drug (or drugs) at a time other than promptly after administration.

An “extended release dosage form” is one that allows at least a twofold reduction in dosing frequency as compared to that drug presented as a conventional dosage form (e.g. as a solution or prompt drug-releasing, conventional solid dosage form).

An “extended release dosage form” is one that allows at least a twofold reduction in dosing frequency as compared to that drug presented as a conventional dosage form (e.g. as a solution or prompt drug-releasing, conventional solid dosage form).

A “modified release dosage form” is one for which the drug release characteristics, time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

“Pulsed release” or “pulsatile release” refers to an initial release of drug, followed by a period of substantially no release, followed by one or more additional releases of drug separated by a period of substantially no release. This does not mean that there are no blood levels of drugs between periods of release.

“Sustained release” or “SR” means the therapeutic pharmaceutical composition is provided in a formulation such that the composition provides an initial therapeutic effect and also an ongoing or additional release of the therapeutic pharmaceutical composition or therapeutic effect over a desired period of time.

A. Inert Substrate

The inert substrate is preferably a water soluble polymer, sugar, or salt, but can be a water-insoluble, or poorly water-soluble material. The substrate can be porous or non-porous. The material may be hydrophilic. The shape and size, and resulting surface area, can be used to manipulate the rate of dissolution of the applied drug. The inert substrate may be spherical, caplet shaped, pellets, beads, particles or crystals.

The substrates can be prepared by wet granulation, spheronization, coacervation, sieving, milling, or spraying, for example, into supercritical fluids. Preferred examples of nonpareils are sucrose or sucrose and starch beads or minispheres.

Excipients such as surfactants can be added to alter water uptake or dissolution properties. These can be blended with or incorporated into pores in the substrate. Solution/dispersion/emulsion of solubility enhancing agents and drug can be applied together or sequentially to inert cores by coating: top or bottom spray (Wurster) or pan coating. Drag and/or solubility enhancing agents can also be applied by electrostatic processes or ink jet technology/microdots.

B. Active Agent(s)

The formulations contain a corticosteroid. Corticosteroids are a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior. Glucocorticoids, such as cortisol, control carbohydrate, fat and protein metabolism and are anti-inflammatory by preventing phospholipid release, decreasing eosinophil action and through a number of other mechanisms. Mineralocorticoids, such as aldosterone, control electrolyte and water levels, mainly by promoting sodium retention in the kidney.

Glucocorticoids can also be artificially made, and are usually referred to as glucocorticoid drugs. Examples of glucocorticoids are prednisone, prednisilone, methylprednisilone, dexamethasone, hydrocortisone, desoximetasone, mometasone furoate, triamcinolone acetonide, triamcinolone acetate, flucinolone acetonide, hydrocortisone valerate, mometasone furoate, cloeortolone privalate, fluticasone propionate, budesonide and fluticasone.

In a preferred embodiment, the active agent is fluticasone or a pharmaceutically acceptable salt thereof such as fluticasone propionate. Fluticasone propionate, also known as S-fluoromethyl-6-α-9-difluoro-11-β-hydroxy-16-α-methyl-3-oxoandrosta-1,4-diene-17-β-carbothioate, 17-propionate, is a synthetic, trifluorinated, corticosteroid having the chemical formula C₂₅H₃₁F₃O₅S. If is a white to off-white powder with a molecular weight of 500.6 g/mol. Fluticasone propionate is practically insoluble in water (0.14 μg/ml), freely soluble in dimethyl sulfoxide and dimethyl-formamide, and slightly soluble in methanol and 95% ethanol. Studies using oral dosing of labeled and unlabeled fluticasone propionate have demonstrated that the oral systemic bioavailability of fluticasone propionate is negligible (<1%), primarily due to incomplete absorption aid presystemic metabolism in the gut and liver.

Fluticasone proprionate is the most widely used pharmaceutically acceptable salt. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric acids; and the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic acids.

Fluticasone propionate is described in British Patent No. 2088877 to Glaxo Group. The compound has potent anti-inflammatory activity and is particularly useful for the treatment of respiratory disorders, particularly asthma. In vitro assays using human lung cytosol preparations have, established fluticasone propionate as a human glucocorticoid receptor agonist with an affinity 18 times greater than dexamethasone, and almost twice that of beclomethasone-17-monopropionate (BMP), the active metabolite of budesonide.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

In another embodiment, the active agent is budesonide. In yet another embodiment, the active agent is a combination of a corticosteroid and 5-aminosalicylic acid.

C. Excipients

Formulations are prepared using pharmaceutically acceptable “carriers” composed of materials that are considered safe and effective and may be administered to an individual without causing undesirable biological side effects or unwanted interactions. The term “carrier” refers to all components present in the pharmaceutical formulation other than the active ingredient or active ingredients. The term “carrier” includes but is not limited to diluents, binders, lubricants, disintegrators, fillers, and coating compositions. The term “carrier” also includes all components of the coating composition, which may include plasticizers, pigments, colorants, stabilizing agents, and glidants.

Examples of suitable coating materials include, but are not limited to, cellulosic polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid, polymers and copolymers, and methacrylic resins that are commercially available under the trade name EUDRAGIT® (Roth Pharma, Westerstadt, Germany), zein, shellac, and polysaccharides. The coating material may contain conventional excipients, such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, macrocrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydoxypropylmethylcellulose, hydroxypropylcellulose, ethyl cellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants ate used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing, carboxylase, sulfonate, and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, “quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The preferred coating weights for particular coating materials may be readily determined by those skilled in the art by evaluating individual release profiles for tablets, beads and granules prepared with different quantities of various coating materials. It is the combination of materials, method, and form of application that produce the desired release characteristics, which one can determine only from the clinical studies.

The coating composition may include conventional additives, such as plasticizers, pigments, colorants, stabilizing agents, gildants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is tale. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Dryers (non-stick agents) have the following properties: they have large specific surface areas, are chemically inert, are free-flowing and comprise fine particles. Because of these properties, they reduce the tack of polymers containing polar comonomers. Examples of dryers are: Alumina, magnesium oxide, kaolin, talc, silica (Aerosils), barium sulfate and cellulose.

Examples of release agents are: esters of fatty acids or fatty amides, aliphatic, long-chain, carboxylic acids, fatty alcohols and esters thereof, montan waxes or paraffin waxes and metal soaps; particular mention should be made of glycerol monostearate, stearyl alcohol, glycerol behenic acid ester, cetyl alcohol, palmitic acid, canauba wax, beeswax etc. The usual proportionate amounts are in the range from 0.05% by weight to 5, preferably 0.1 to 3, % by weight based on the copolymer.

Substances suitable as plasticizers would ordinarily have a molecular weight between 100 and 20 000 and contain one or more hydrophilic groups in the molecule, e.g. hydroxyl, ester or amino groups. Citrates, phthalates, sebacates, castor oil are suitable. Examples of suitable plasticizers are alkyl citrates, glycerol esters, alkyl phthalates, alkyl sebacates, sucrose esters, sorbitan esters, dibutyl sebacate and polyethylene glycols 4 000 to 20 000. Preferred plasticizers are tributyl citrate, triethyl citrate, triethyl acetylcitrate, dibutyl sebacate and diethyl sebacate. The amounts used are between 1 and 35, preferably 2 to 10, % by weight based on the (meth)acrylate copolymer.

Solubility promoting agents that can be incorporated into the drug-polymer layer include those described in the examples as well as polyethylene oxide, cyclodextrins, polyvinylpyrollidone, d-alpha-tocopheryl polyethylene glycol 1000 succinate (TPGS), vitamin E, lipids, triglycerides, bile acids.

D. Inner Drug Coating

As used herein, the drug may incorporate or be coated with a polymer, copolymer, or blend thereof of one or more polymers and/or copolymers. Unless otherwise specified, reference to inner drug coating refers to either embodiment.

In a preferred embodiment, the inner drug coating consists of a copolymer or a mixture of copolymers composed of 85 to 98% by weight free-radical polymerized C1-C4-alkyl esters of acrylic or methacrylic acid and 15 to 2% by weight (meth)acrylate monomers with a quaternary ammonium group in the alkyl radical.

Suitable (meth)acrylate copolymers are disclosed, for example, in EP-A 181 515 or DE 1 617 751. These are polymers which are soluble or swellable independently of the pH are suitable for pharmaceutical coatings. A possible production process to be mentioned is bulk polymerization in the presence of a free-radical initiator dissolved in a monomer mixture. The polymer can also be produced by solution or precipitation polymerization. The polymer obtained in this way is in the form of a fine powder, which is achievable in the case of bulk polymerization by grinding, and in the case of solution and precipitation polymerization, for example, by spray drying.

Preferred C1- to C4-alkyl esters of acrylic or methacrylic acid are methyl acrylate, ethyl acrylate, butyl acrylate, butyl methacrylate and methyl methacrylate. The particularly preferred (meth)acrylate monomer with quaternary ammonium groups is 2-trimethylammoniumethyl methacrylate chloride. Suitable copolymers include, but are not limited to, copolymers containing 93 to 98% by weight free-radical polymerized C1- to C4-alkyl esters of acrylic or methacrylic acid and 7% to 2% by weight 2-trimethylammoniumethyl methacrylate chloride. Examples of possible contents in this case are 50-70% by weight methyl methacrylate, 20-40% by weight ethyl acrylate.

A corresponding copolymer is composed, for example, of 65% by weight methyl methacrylate, 30% by weight ethyl acrylate and 5% by weight 2-trimethylammoniumethyl methacrylate chloride (EUDRAGIT® RS).

A further suitable copolymer can be produced, for example, from 85% to 93% by weight free-radical polymerized C1- to C4-alkyl esters of acrylic or methacrylic acid and 15% to 7% by weight 2-trimethylammoniumethyl methacrylate chloride, Examples of possible contents in this ease are 50-70% by weight methyl methacrylate, 20-40% by weight ethyl acrylate.

A suitable copolymer is composed of 60% by weight methyl methacrylate, 30% by weight ethyl acrylate and 10% by weight 2-trimethylammoniumethyl methacrylate chloride (EUDRAGIT® RL).

The proportionate amount of the inner coating should be in the range from 2 to 20% by weight based on the core with the active ingredient. It is favorable to use both the above-mentioned copolymer types simultaneously, preferably those with 5 and with 10% by weight 2-trimethylammoniumethyl methacrylate chloride (EUDRAGIT® RS and EUDRAGIT® RL) in a mixture. The ratio of the mixture can be, for example, 20:1 to 1:20, preferably 10:1 to 1:10.

E. Outer Coating

The outer coating is an enteric coating. The outer coating contains a copolymer composed of 75% to 95% by weight, preferably 85% to 95% by weight free-radical polymerized C₁ to C₄-alkyl esters of acrylic or methacrylic acid and 5% to 25% by weight, preferably 5% to 15% by weight (meth)acrylate monomers with an anionic group in the alkyl radical. C₁-C₄ alkyl esters of acrylic or methacrylic acid are, in particular, methyl methacrylate, ethyl methacrylate, butyl methacrylate, methyl acrylate, ethyl acrylate and butyl acrylate. A (meth)acrylate monomer with an anionic group in the alkyl radical can be, for example, acrylic acid, but preferably methacrylic acid.

Particularly suitable (meth)acrylate copolymers are those composed of 10 to 30% by weight methyl methacrylate, 50 to 70% by weight methyl acrylate and 5 to 15% by weight methacrylic acid (EUDRAGIT® FS type).

The following general disclosure relates to the use of excipients and polymers to produce immediate, sustained, delayed, or pulsed formulations. The can be achieved through manipulation of the drug-binder/inner drug coating and outer coating layers.
Immediate Release Formulations

Typical immediate release formulations include compressed tablets, gels, films, coatings, liquids and particles that can be encapsulated, for example, in a gelatin capsule. Many methods for preparing coatings, covering or incorporating drugs, are known.

The immediate release dosage unit of the dosage form, i.e., a tablet, a plurality of drug-containing beads, granules, or particles, or an outer layer of a coated core dosage form, contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads). A preferred method for preparing immediate release tablets (e.g., as incorporated into a capsule) is by compressing a drug-containing blend, e.g., blend of granules, prepared using a direct blend, wet-granulation or dry-granulation process. Immediate release tablets may also be molded rather than compressed, starting with a moist material containing a suitable water-soluble lubricant. However, preferred tablets described herein are manufactured using compression rather than molding. A preferred method for forming immediate release drug-containing blend is to mix drug particles directly with one or more excipients such as diluents (or fillers), binders, disintegrants, lubricants, glidants, and colorants. As an alternative to direct blending, a drug-containing blend may be prepared by using a wet-granulation or dry-granulation process. Beads containing the active agent may also be prepared by airy one of a number of conventional techniques, typically starting from a fluid dispersion. For example, a typical method for preparing drug-containing beads involves blending the active agent with conventional pharmaceutical excipients such as microcrystalline cellulose, starch, polyvinylpyrrolidone, methylcellulose, talc, metallic stearates, and silicone dioxide. The admixture is used to coat a bead core such as a sugar sphere (e.g., “non-pareil”) having a size of approximately 20 to 60 mesh.

An alternative procedure for preparing drug beads is by blending the drug with one or more pharmaceutically acceptable excipients, such as microcrystalline cellulose, lactose, cellulose, polyvinyl pyrrolidone, talc, magnesium stearate, and a disintegrant, extruding the blend, spheronizing the extrudate, drying and optionally coating the bead to form immediate release beads.

Extended or Sustained Release Dosage Forms

Extended release formulations are generally prepared as diffusion or osmotic systems, for example, as described in “Remington—The Science and Practice of Pharmacy”, 20th Ed., Lippincott (Williams & Wilkins, Baltimore, Md., 2000). A diffusion system typically consists of one of two types of devices, reservoir and matrix, which are well-known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and Carbopol® 934, and polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeability and high permeability coating materials in suitable proportion.

An immediate release portion can be added to the extended release system by means of either applying an immediate release layer on top of the extended release core using coating or compression processes or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such, as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed, with a wax material and either spray-congealed or congealed and screened and processed.

Delayed Release Dosage Forms

Delayed release dosage formulations are created by coating a solid dosage form with a film, of a polymer which is insoluble in the acid environment of the stomach, and soluble in the neutral environment of the small intestine.

The delayed release dosage units can be prepared, for example, by coating a drug or a drag-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually wafer-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon.

Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0and above), EUDRAGIT® S (soluble at pH 7.0 and above, as a result of a higher degree of esterification), and EUDRAGIT® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers; pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

Alternatively, a delayed release tablet may be formulated by dispersing the drug within a matrix of a suitable material such as a hydrophilic polymer or a fatty compound. Suitable hydrophilic polymers include, bit are not limited to, polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

Pulsed Release Dosage Forms

A pulsed release dosage form is one that mimics a multiple dosing profile without repeated dosing and typically allows at least a twofold reduction in dosing frequency as compared to the drug presented as a conventional dosage form (e.g. as a solution or prompt drug-releasing, conventional solid dosage form). A pulsed release profile is characterized by a time period of no release (lag time) or reduced release followed by rapid drug release.

Each dosage form contains a therapeutically effective amount of active agent. In one embodiment of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 30 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

For dosage forms mimicking a three times daily dosing profile, approximately 25 wt. % to 40 wt. % of the total amount of active agent in the dosage form is released in the initial pulse, and approximately 25 wt. % to 40 wt. % of the total amount of active agent in the dosage form is released in each of the second and third pulses. For dosage forms that mimic a three times daily dosing profile, release of the second pulse preferably takes place approximately 3 hours to 10 hours, and more preferably approximately 4 to 9 hours, following oral administration. Release of the third pulse occurs about 2 hours to about 8 hours following the second pulse, which is typically about 5 hours to approximately 18 hours following oral administration.

The dosage form can be a closed capsule housing at least two drug-containing dosage units, each dosage unit containing one or more compressed tablets, or may be contain a plurality of beads, granules or particles, providing that each dosage unit has a different drug release profile. The immediate release dosage unit releases drug substantially immediately following oral administration to provide an initial dose. The delayed release dosage unit releases drug approximately 3 hours to 14 hours following oral administration to provide a second dose. Finally an optional second delayed release dosage unit releases drug about 2 hours to 8 hours following the release of the second dose, which is typically 5 hours to 18 hours following oral administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of beads, granules or particles that release drug substantially immediately following oral administration to provide an initial dose. The delayed release dosage unit comprises a plurality of coated beads or granules, which release drug approximately 3 hours to 14 hours following oral administration to provide a second dose.

An optional second delayed release dosage unit contains coated beads or granules that release drug about 2 to 8 hours following administration of the initial delayed release dose, which is typically 5 to 18 hours following oral administration. The beads or granules in the delayed release dosage unit(s) are coated with a bioerodible polymeric material. This coating prevents the drug from being released until the appropriate time, i.e., approximately 3 hours to less than 14 hours following oral administration for the delayed release dosage unit and at least 5 hours to approximately 18 hours following oral administration for the optional second delayed release dosage unit. In this dosage form the components may be admixed in the tablet or may be layered to form a laminated tablet.

Another dosage form is a tablet having a drug-containing immediate release dosage unit, a delayed release dosage unit, and an optional second delayed release dosage unit, wherein the immediate release dosage unit contains an outer layer that releases the drug substantially immediately following oral administration. The arrangement of the remaining delayed release dosage(s), however, depends upon whether the dosage form is designed to mimic twice daily dosing or three times daily dosing.

In the dosage form mimicking twice daily dosing, the delayed release dosage unit contains an inner core that is coated with a bioerodible polymeric material. The coating applied such that release of the drug occurs approximately 3 hours to less than 14 hours following oral administration. In this form, the outer layer completely surrounds the inner core.

In the dosage form mimicking three times a day dosing, the (first) delayed release dose contains an internal layer that releases drug approximately 3 hours to less than 14 hours following oral administration. This internal layer is surrounded by the outer layer. The second delayed release dosage unit generally comprises an inner core that releases the drug at least 5 hours to approximately 18 hours following oral administration. Thus, the layers of this tablet (starting from the external surface) contain an outer layer, an internal layer and an inner core. The inner core contains delayed release beads or granules. Furthermore, the internal layer contains the drug coated with a bioerodible polymeric material. Alternatively, in this particular dosage form mimicking three times a day dosing, both the delayed release dosage unit and second delayed release dosage units are surrounded by an inner layer. This inner layer is free of active agent. Thus, the layers of this tablet (starting from the external surface) contain an outer layer, inner layer and an admixture of the delayed release dosage units. The first delayed release pulse occurs once the inner layer is substantially eroded thereby releasing the admixture of the delayed release dosage units. The dose corresponding to the (first) delayed release dosage unit is released immediately since the inner layer has prevented access to this dose for the appropriate time, e.g., from approximately 3 hours to 10 hours. The second delayed release dose, however, is formulated to effectively delay release for at least 5 hours to approximately 18 hours following oral administration.

For formulations mimicking twice daily dosing, it is preferred that the delayed release dose is released approximately 3 hours to up to 14 hours, more preferably approximately 5 hours to up to 12 hours, following oral administration. For formulations mimicking three times daily dosing, it is preferred that the (first) delayed release dose is released approximately 3 to 10 hours, preferably 4 hours to 9 hours, following oral administration. For dosage forms containing a third dose, the third dose (i.e., the second delayed release dose) is released at least 5 hours to approximately 18 hours following oral administration.

In still another embodiment, a dosage form is provided which contains a coated cure-type delivery system wherein the outer layer contains an immediate release dosage unit containing an active agent, such that the active agent therein is immediately released following oral administration; an intermediate layer there under which surrounds a core; and a core which contains immediate release beads or granules and delated release beads or granules, such that the second dose is provided by the immediate release beads or granules and the third dose is provided by the delayed release beads or granules.

Drug complexes ate generally prepared by complexing the drug with a pharmaceutically acceptable ion-exchange resin. The complex is formed by reaction of a functional group of the drug with a functional group on the ion exchange resin. Drug is released by exchanging with appropriately charged ions within the gastrointestinal tract.

Ion-Exchange Resins

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound salt forming groups may be strongly acidic (e.g., sulfonic acid or sulfuric acid) or weakly acidic (e.g., carboxylic acid). In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975).

Resins include Amberlite® IRP-69 (Rohm and Haas) INDION® 224, INDION® 244, and INDION® 254 (Ion Exchange (India) Ltd.). These resins are sulfonated polymers composed of polystyrene cross-linked with divinylbenzene. Any ion-exchange resins currently available and those that should became pharmaceutically acceptable and available in the future can also be used. Commercial sources of ion exchange resins that are either pharmaceutically acceptable or may become pharmaceutically acceptable in the future include, out are not limited so, Rohm and Haas, The Dow Chemical Company, and Ion Exchange (India) Ltd.

The size of the ion-exchange particles should be less than about 2 millimeter, more preferably below about 1000 micron, more preferably below about 500 micron, and most preferably below about 150 micron. Commercially available ion-exchange resins (Amberlite® IRP-69, INDION® 244 and INDION® 254) have a particle size range less than 150 microns.

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. No. 4,221,778 to Raghunathan; U.S. Pat. No. 4,894,239 to Nonomura; and U.S. Pat. No. 4,996,047 to Kelleher.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt, form); and (d) resin (H-form) plus drag (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

The resin-drug complexes can be incorporated into tablets, capsules, beads, films, coatings or particles. The resin-drug complexes or particles containing the complexes can also be suspended in a liquid such as a syrup. The complexes or particles can also be coated with a material such as an enteric coating or barrier to alter release properties. Complexes with different coatings, or mixture of uncoated with coated complexes or particles, can be used to create mixtures with different release properties.

### II. Methods of Manufacturing

As will be appreciated by those skilled in the art and as described in the pertinent texts and literature, a number of methods are available for preparing drug-containing tablets, beads, capsules, granules or particles, films and coatings that provide a variety of drug release profiles. Such methods include, but are not limited to, coating a drug or drug-containing composition with an appropriate coating material, increasing drug particle size, placing the drug within a matrix of excipient and other fillers, coating the material with an enteric coating, and forming complexes of the drug with a suitable complexing agent such as an ion-exchange resin.

Coatings can be applied aqueous or organic solutions or suspensions. Film coatings are typically thin barrier films, providing protection or color to the particles or tablets. Active ingredient can be incorporated into the coating. Coatings may be formed of lipids or by the hot melting of polymers. This provides coatings of between 25 and several hundred microns in thickness, which protect against moisture. No evaporation of solvents is required. Sugar coatings are generally between 0.5 and 2 mm. These are used to provide taste masking and sealing, as well as for protection and coating of temperature-sensitive and fragile products. The coating is applied by spraying of a syrup onto the particles.

These sprayed coatings can vary between approximately 5 microns and 50 microns or more. Coatings can be applied as polymeric solutions or sprays by fluidized bed reactors, by spray coating (top spray, Wurster coating—bottom spray), or tangential spray—rotor pellet coating), or drum or pan coaters. Top spray coatings are used for general coatings including enteric coatings. Particles are fluidized in the flow of heated air, which is introduced into the product container, an the coating liquid is sprayed into the fluid bed from above. Drying takes place as the particles move upward. Bottom spraying is particularly suitable for controlled release of active ingredients. In the Wurster process, a complete sealing of the surface can be achieved with a low usage of coating substance. The spray nozzle is fitted in the base plate resulting in a spray pattern that is concurrent with the air feed. By using a Wurster cylinder and a base plate with different perforations, the particles to be coated are accelerated inside the Wurster tube and fed through the spray cone concurrently. As the particles continue traveling upwards, they dry and fall outside the Wurster tube back towards the base plate. They are guided from the outside back to the inside of the tube where they are once again accelerated by the spray. This produces an extremely even film. Particles of different sizes are evenly coated. Particularly suitable for protective coatings/color coatings where the product throughput rates are high. For continuous fluid bed coatings, the product is continuously fed into one side of the machine and is transported onwards via the sieve bottom by means of the air flow. Depending on the application, the system is sub-divided into predicating zones, spray zones and drying zones whereby spraying, can take place from below in the form of a bottom spray. The dry, coated particles are continuously extracted. Tangential spray coatings (Rotor pellet coating) are ideal for coatings with high solid content. The product is set into a spiral motion by means of a rotating base plate, which has air fed into the powder bed at its edge. The spray nozzle is arranged tangentially to the rotor disc and also sprays concurrently into the powder bed. Very thick film layers can be applied by means of the rotor method. Tablets and dragees are coated using drum or pan coats. These am typically for the application of protective films or taste masking.

Powder particles can be agglomerated in a fluid bed to buildup powder granulates, typically in the size range of 0.2 and 2.5 mm. The powder is moistened in order to form liquid bridges between the particles. The spray liquid can be either water or an organic solvent which dissolves the powder or a binder. The moistened granulates are dried and cooled. These have a low bulk density and are highly water soluble. Wet granulation is used to build up granulates from powder. These are generally denser and more mechanically stable particles than fluid bed granulates. These produce grains between 0.1 and 10 mm. Wet granulation in a vertical granulator is the classical method for building up granulates from powder. In this process, powder is led to a product container and then moistened or sprayed with molten material in older to increase the cohesive forces. The liquid can be water or an organic solvent, if necessary with a binder. At the same time, the ingredients are mixed together vigorously. Denser granulates are formed than in the case of in the fluid bed. The products are highly suitable for making into tablets, compact, with low hydroscopicity. Spray granulation is the drying of liquids (solutions, suspensions, melts) while simultaneously building up granulates.

Spray granulates are denser and harder in comparison with agglomerates. The spray granulation of different starting materials that have been mixed in the liquid phase produces granulates, in which the starting materials are very evenly distributed. If the process is set up correctly, liquids can also be encapsulated in a fixed matrix in this way.

If the matrix material is dissolved in the liquid phase, the granulates are made by means of spray granulation. If the matrix material is presented in the form of powder, the granulates are made by means of wet granulation. This encapsulation process is mainly applied in the food industry. If necessary, a protective coating can be applied to the spray granulates in an additional step.

Blending is the dry mixing of ingredients to produce an uniform distribution of components. In solid processes, various individual products of different density and concentration and in different amounts are often admixed to form a homogeneous mixture. In the pharmaceutical area, very different quantities and proportions of active and auxiliary ingredients (corn starch, lactose, PVP, etc.) are mixed together. Specific auxiliary materials such as lubricants or flavorings may also be added. Mixing may be necessary in different process sections. For instance, compression aids, flow controlling media and external phases are added following the granulation process and before compression.

Direct pelletizing is the manufacture of pellets directly from powder. Pellets can be prepared by building op layer by layer around a starting core, or a round pellet can be extruded by spheronizing. Spray granulation can also be used for build-up of liquid particles. In direct pelletizing, pellets are manufacture directly from powder with a binder or solvent. This is a fast process and yields compact round pellets, which have a higher density than spray granulates and agglomerates. Pellet diameters are between 0.2 and 1.2 mm. Pellets can be made into tablets or used to fill capsules. Pelletizing, by layering, results in a layer by layer build-up of material around a given starting core. This is ideal for forming round pellets with separate layers of powder coatings and/or active agent. The layers are densely applied due to the movement of the pellets in the rotor. Thick layers can be applied to the starting grains, which allow large amounts of active to be incorporated. These have a higher density than spray granulates and agglomerates. Typical diameters are between 0.6 and 2.5 mm. In spheronizing, round pellets are formed from irregular wet granulates and extruded products. The moist granulates or extruded products are fed onto a rotating/pelletizing plate. The surface is smoothed due to the intensive rolling movement and spherical pellets are produced due to the intensive rolling movement. This results in narrow particle size distribution and good flow behavior. Pellets have a higher density than spray granulates and agglomerates. Typical particle diameters are between 0.5 and 2.5 mm. Spray granulation is the drying of liquids (solutions, suspensions, melts) while simultaneously building up of granulates. These are denser and harder than agglomerates and have a size between 0.2 and 5 mm.

For detailed information concerning materials, equipment and processes for preparing tablets and delayed release dosage forms, see Pharmaceutical Dosage Forms: Tablets, eds, Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995). A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere for so-called “non-pareil”) having a size of approximately 20 to 60 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, and a disintegrant, extruding the blend, spheronizing the extrudate, drying and optionally coating to form the immediate release beads.

The formulations may be prepared as described in references such as “Pharmaceutical dosage form tablets”, Eds. Liberman et. al. (New York, Marcel Dekker, Inc., 1989), “Remington—The Science and Practice of Pharmacy”, 20th Ed., Lippincott (Williams & Wilkins, Baltimore, Md., 2000), and “Pharmaceutical dosage forms and drug delivery systems”, 6th Ed., Ansel et. al., (Media, Pa.: Williams and Wilkins, 1995) which provides information on carriers, materials, equipment and process for preparing tablets and capsules and delayed release dosage forms of tablets, capsules, and granules.

In a preferred embodiment, the proportionate amount of the outer coating should be in the range from 10 to 50% by weight based on the weight of the core with the active ingredient and the inner coating. The copolymers are obtained in a manner known per se by free-radical bulk, solution, bead or emulsion polymerization. Before processing, they must be brought to a desired particle size range by suitable grinding, drying or spraying processes. This can take place by simple crushing of extruded and cooled pellets or hot cut. Preference is given to emulsion polymerization in aqueous, phase in the presence of water-soluble initiators and (preferably anionic) emulsifiers.

The emulsion polymer is preferably produced, and used in the form of a 10 to 50 percent by weight, in particular 30 to 40 percent by weight, aqueous dispersion. Partial neutralization of the methacrylic acid units is not necessary for processing; if is, however, possible, for example to the extent of 5 or 10 mol %, if thickening of the coating agent dispersion is desired. The weight-average size of the latex particles is ordinarily 40 to 100 nm, preferably 50 to 70 nm, which ensures a viscosity of below 1 000 mPa, which is favorable for processing.

The minimum film-forming temperature (MFT; DIN 53 455) is between 0 and 25° C. for most of the coating agents, so that processing is possible at room temperature without added plasticizer. The elongation at break of the films, measured in accordance with DIN 53 455, is ordinarily 50% or more with a triethyl citrate content not exceeding 10% by weight.

### III. Dosage Forms and Methods of Administration

The compositions described herein can be administered in the form of a coated tablet, a tablet composed of compressed pellets or pellets which are packed in a soft or hard capsule, for example made of gelatin, starch or cellulose derivatives.

The pharmaceutical formulation may be in any suitable form, including liquid suspensions and solid dosage forms. Formulations with different drug release mechanisms can be combined in a final dosage form including single or multiple units. Examples of multiple units include multilayer tablets, capsules containing tablets, beads, granules, etc, in a solid or liquid form.

The formulations are administered orally to individuals in need thereof, in a therapeutically effective amount to alleviate one or more symptoms of a disorder, usually a gastrointestinal disorder such as Crohn's disease or ulcerative colitis. In a preferred embodiment, the dose of fluticasone proprionate is from about 1 mg to about 25 mg.

This may also be packaged to provide for an escalating or tapering dosage.

### EXAMPLES

The present invention will be further understood by reference to the following non-limiting examples.

Fluticasone propionate has a solubility of 0.14 μg/ml in water. The solubility of fluticasone from pellets in a multilayer system can be increased substantially to approximately 1-10 μg/ml in water by preparing solid dispersions using polymeric carriers with acidic groups. Pellets were formulated by layering solid dispersions of micronized fluticasone with solubility enhancing agents onto the inert cores. Trials were carried out by forming solid dispersions of drug and polymer in different ratios and these dispersions were loaded onto non-pareil seeds (Sugar spheres NF) using a fluid bed coater. Different polymers used were Kollidon® VA 64, EUDRAGIT® L100-55, EUDRAGIT® L30-D55.

The solubility of fluticasone propionate was determined by carrying out dissolution in accordance with USP-29 monograph dissolution apparatus II (paddle) with 150 revolutions per minutes in phosphate buffer of pH 7.5 with 500 ml dissolution volume for a period of 24 hours. Sample equivalent to 12.5 mg of fluticasone propionate was weighed for each determination. Detection was done by means of HPLC using method described in USP-29.

### Example 1

### Dissolution Studies of Fluticasone Propionate

Kollidon® VA 64 was dissolved in a sufficient amount of isopropyl alcohol with stirring. Fluticasone propionate was added slowly to the Kollidon® solution. The drug suspension was sonicated for uniform distribution of the drug. The drug suspension was kept under constant stirring with the help of a magnetic stirrer through out the experiment. The above drug suspension was loaded onto non-pareil seeds (710-850 μm) in a fluid bed coater using bottom spray.

The drug to polymer ratio was 1:4. With the use of Kollidon® VA 64 as a polymeric carrier for fluticasone propionate in the ratio of 1:4, there was an increase in the solubility.

### Example 2

### Dissolution Studies of Fluticasone Propionate

Glyceryl monostearate was added to hot water (80-85° C.) and homogenized. This suspension was then cooled to room temperature. Triethyl citrate and fluticasone propionate were added slowly into the above suspension and homogenized. This drug suspension was mixed in to the EUDRAGIT® L30D-55, which was kept under stirring using magnetic stirrer. The suspension thus formed was filtered through 100 mesh and kept under stirring with the help of a magnetic stirrer through out the experiment. This aqueous drug dispersion was loaded onto non-pareil seeds (710-850 μm) in a fluid bed coater using bottom spray.

The results of the solubility of pellets with a drug to polymer ratio of 1:4 were as follows:

| Sr. | Drug:Polymer | | Mean solubility |
|---|---|---|---|
| No. | Ratio | Solubility (μg/ml) | (μg/ml) |
| 1 | 1:4 | 0.377-0.741 | 0.559 |

With the use of Eudragit® L 30D 55 as a polymeric carrier for fluticasone propionate in a drug to polymer ratio of 1:4, there was a 6 fold increase in the solubility.

### Example 3

### Dissolution Studies of Fluticasone Propionate

Triethyl citrate and Eudragit® L 100-55 was dispersed in isopropyl alcohol and this solution was kept under stirring. Fluticasone propionate was dispersed in isopropyl alcohol. Both theses solutions are mixed together under constant stirring and the final suspension was kept under stirring through out the experiment. This drug dispersion was loaded onto ion-pareil seeds (710-850 μm) in a fluid bed coater.

The results of the solubility of the pellets with a drug to polymer ratios of 1:4, 1:6 and 1:8 were as follows:

| Sr. | Drug:Polymer | | Mean solubility |
|---|---|---|---|
| No. | Ratio | Solubility (μg/ml) | (μg/ml) |
| 1 | 1:4 | 1.897-2.225 | 2.075 |
| 2 | 1:6 | 2.252-2.547 | 2.462 |
| 3 | 1:8 | 2.377-3.400 | 2.814 |

With the use of Eudragit® L 100 55 as a polymeric carrier for fluticasone propionate in the ratio of 1:4, there was a 20 fold increase in the solubility.

At the drug to polymer ratio of 1:6, the solubility was found to have increased almost 24 times.

When the polymer content was increased to a ratio of 1:8 (drug to polymer), the solubility obtained was almost 28 times more than the pellets formulated without any polymeric carrier.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A multi layer pharmaceutical composition comprising:
a. a core containing a solid dispersion of one or more active pharmaceutical agents and preferably one or more solubility enhancing agents on an inert substrate,
b. an inner coating on the core, which may incorporate active agent or be layered onto active agent, the inner coating comprising one or more controlled release polymers, and
c. an outer coating of one or more pH sensitive polymers.

2. The multilayer pharmaceutical composition of claim 1 wherein the multilayer pharmaceutical product releases less than 5% of the active agent during the first 2 hours of a USP release test and provides enhanced solubility and dissolution of the active agent when released into gastrointestinal fluids.

3. The multilayer pharmaceutical composition of claim 1 wherein the active agent is a corticosteroid.

4. The multilayer pharmaceutical composition of claim 3 wherein the active agent is fluticasone or a pharmaceutically acceptable salt thereof.

5. The multilayer pharmaceutical composition of claim 4, wherein the active agent is fluticasone proprionate.

6. The multilayer pharmaceutical composition of claim 5, wherein the dose of fluticasone proprionate is from about 1 mg to about 25 mg.

7. The multilayer pharmaceutical composition of claim 1 wherein the active agent is budesonide and the budesonide is incorporated into the controlled release polymer.

8. The multilayer pharmaceutical composition of claim 1, wherein the active agents is a combination of a corticosteroid and 5-aminosalicylic acid.

9. The multilayer pharmaceutical composition of claim 1, wherein the inert core is a non-pareil seed, salt, or polymer particle.

10. The multilayer pharmaceutical composition of claim 9, wherein the non-pareil seed is sugar sphere having a diameter from about 710 to about 850 microns.

11. The multilayer pharmaceutical composition of claim 1 comprising solubility enhancing agents selected from the group of synthetic polymers consisting of (meth)acrylate copolymers composed of 40 to 60% by weight methacrylic acid and 60 to 40% by weight ethyl acrylate, copolymers consisting of 65% by weight methyl methacrylate, 30% by weight ethyl acrylate and 5% by weight 2-trimethylammoniumethyl methacrylate chloride; copolymers consisting of 60% by weight methyl methacrylate, 30% by weight ethyl acrylate and 10% by weight 2-trimethylammoniummethyl methacrylate chloride; copolymesr consisting of 60% by weight vinyl pyrrolidine and 40% by weight vinyl acetate; and combinations thereof.

12. The multilayer pharmaceutical composition of claim 1, wherein the solubility enhancing agents are selected from the group consisting of polyethylene oxide, cyclodextrins, polyvinylpyrrolidone, d-alpha-tocopheryl polyethylene, glycol 1000 succinate (TPGS), vitamin E, lipids, triglycerides, bile acids, and combinations thereof.

13. The multilayer pharmaceutical composition of claim 1, wherein the core further comprises one or more pharmaceutically acceptable excipients.

14. The multilayer pharmaceutical composition of claim 13, wherein the one or more pharmaceutically acceptable excipients are selected from the group consisting of alkyl citrates, glycerol esters, alkyl phthalates, alkyl sebacates, sucrose esters, sorbitan esters, dibutyl sebacate and polyethylene glycols 4,000 to 20,000

15. The multilayer pharmaceutical composition of claim 1, wherein the ratio of drug to solubility enhancing agents is 1:2-1:8.

16. The multilayer pharmaceutical composition of claim 1, wherein the inner coating comprised one or more polymers consisting of 93 to 98% by weight C1- to C4-alkyl esters of acrylic or methacrylic acid and 2 to 7% by weight 2-trimethylammoniumethyl methacrylate chloride.

17. The multilayer pharmaceutical composition of claim 1, wherein the outer coating polymers are preferably (meth)acrylate copolymers composed of 10 to 30% by weight methyl methaerylate, 50 to 70% by weight methyl acrylate and 5 to 15% by weight methacrylic acid.

18. The multilayer pharmaceutical product of claim 1, wherein the inner coating is from 2 to 50% by weight of the core.

19. The multilayer pharmaceutical product of claim 1, wherein the outer coating is from 5 to 50% by weight based on the weight of the core and the inner coating.

20. The multilayer pharmaceutical composition claimed in 1 where the solubility of fluticasone proprionate is 0.14-10 μg/ml

21. The multilayer pharmaceutical composition product as in claim 1, wherein said multilayer pharmaceutical product is in the form of pellets, tablets compressed from pellets or pellets packed into capsules.

22. The multilayer pharmaceutical composition of 1 comprising a mixture of different compositions of inner and outer polymers.

23. The multilayer pharmaceutical composition of 1 releasing pharmaceutically active agent in the distal ileum and/or colon.

24. A method of making the multilayer pharmaceutical composition of claim 1 comprising loading the core with a dispersion causing fluidized bed coating with bottom spray.

25. A method of treating a person in need thereof comprising administering the composition of claim 1.

26. The method of claim 30 comprising administering the composition for the treatment of inflammatory bowel disease.

27. The method of claim 30 wherein the indications for treatment are Crohn's disease or Ulcerative Colitis.

28. A kit comprising the composition of claim 1 packaged to provide for an escalating or tapering dosage.
